(19) United States
(12) Patent Application Publication
(10) Pub. No.: US 2008/0188834 A1
(43) Pub. Date: Aug. 7, 2008

(54) Pulmonary secretion mobilization device

(76) Inventor: Lisa A. Balducci, Ringoes, NJ (US)

Correspondence Address: Kenneth P. Glynn, 24 Mine Street, Flemington, NJ, 08822, US

(21) Appl. No.: 11/701,113

(22) Filed: Feb. 1, 2007

(51) Int. Cl. A61M 1/00

(52) U.S. Cl. 604/540

(57) ABSTRACT

A pulmonary secretion mobilization device includes: (a.) a cup structure having a hollow inside volume and air impermeable walls and base, the cup structure having a rim in a single flat plane; (b.) an impact-receiving continuous ring connected to the rim, the ring being selected from the group consisting of a gas-containing flexible tube, a liquid-containing flexible tube, a flexible foam ring, a semi-flexible foam ring and an integral skin foam ring; and, (c.) a handle connected to the base. The pulmonary secretion mobilization device cup structure rim may have a footprint of a circle, an oval, a square, a rectangle, polygon or irregular and may be glow-in-the-dark.

### BACKGROUND OF INVENTION

a. Field of Invention

The invention relates generally to a device and methodology for assisting in the loosening of undesired pulmonary secretion that is lodged in a lung or lungs of a person or other mammal. It is an alternative or supplement to conventional medicinal treatments.

b. Description of Related Art

No prior art literature was found to relate to devices and methodologies for patting of lung congested patients to loosen undesired pulmonary secretion. The following patent relates to patting of the underside of a crib:

U.S. Pat. No. 4,951,331 to Fred A. Pereira describes a device for imparting a pat to the underside of an infant crib for helping to soothe or induce sleep in a baby or child. The device includes a frame that sits on the floor beneath the crib. The frame includes a reciprocating means attached to the frame and a “T” bar structure, which has a horizontal member with two ends that have apertures therein and a vertical member attached to the horizontal member midway between the two end. A pair of elongated vertical rods is adjustably attached to each end of the horizontal member by wing nuts. Attached to the top of the vertical rod is a resilient cup-like structure that contracts the underside of mattress when the device is electrically connected to a power source. While this device does have a pair of cups to pat the underside of a crib, it is not a portable device with a handle, as in the present invention, and it is not utilized nor capable of being utilized in accordance with the invention purposes set forth herein.

Notwithstanding the prior art, the present invention is neither taught nor rendered obvious thereby.

### SUMMARY OF INVENTION

The present invention relates to a pulmonary secretion mobilization device that includes: (a.) a cup structure having a hollow inside volume and air impermeable walls and base, the cup structure having a rim in a single flat plane; (b.) an impact-receiving continuous ring connected to the rim, the ring being selected from the group consisting of a gas-containing flexible tube, a liquid-containing flexible tube, a flexible foam ring, a semi-flexible foam ring and an integral skin foam ring; and, (c.) a handle connected to the base.

The handle may be any type of handle that is connectable or connected to the cup structure without interfering with the functionality of the cup structure and the ring. In some preferred embodiments, the handle is a knob handle and in some other preferred embodiments, the handle is an extended handle protruding outwardly from the base. In some preferred embodiments of the present invention pulmonary secretion mobilization device, the handle is an extended rod that is at least 3 inches in length.

In some preferred embodiments of the present invention pulmonary secretion mobilization device, the cup structure has an imaginary central axis and the handle is an extended handle positioned at an angle of about 60 degrees to about 90 degrees measured downwardly from the imaginary central axis.

In some preferred embodiments of the present invention pulmonary secretion mobilization device, the cup structure is symmetrical relative to an imaginary central axis.

In some preferred embodiments of the present invention pulmonary secretion mobilization device, the cup structure rim has a footprint selected from the group consisting of a circle, an oval, a square, a rectangle and polygon having at least 5 sides. Other shapes could be irregular, artistic, or stylized or representative. Random contour shapes, animal outlines, famous cartoon character shapes and images, or any other shapes and images could be used, e.g., a Mickey Mouse head or a stylized heart, with or without two or three dimensional details may entice a child to welcome rather than unjustifiably fear the concomitant methodology.

In some preferred embodiments of the present invention pulmonary secretion mobilization device, the ring is a gas-containing flexible tube made of plastic and containing air.

In some preferred embodiments of the present invention pulmonary secretion mobilization device, the ring has a height of about 3/16 inch to ½ inch as measured perpendicularly from the rim.

In some preferred embodiments of the present invention pulmonary secretion mobilization device, the ring is a urethane foam ring.

In some preferred embodiments of the present invention pulmonary secretion mobilization device, the entire device, i.e. the cup structure, the ring and the handle, may be formed in a single mold with integral skin urethane wherein the ring area is heated to expand the urethane to foam with a skin to make it flexible and air impervious, and the remainder of the device is micro-porous or solid urethane that is air impervious.

In some preferred embodiments of the present invention pulmonary secretion mobilization device, the device, or at least a portion of it, is made of glow-in-the-dark materials or coatings.

In some preferred embodiments of the present invention pulmonary secretion mobilization device, the device includes: (a.) a plastic cup structure having a hollow inside volume and air impermeable walls and base, the cup structure having a rim in a single flat plane; (b.) an impact-receiving continuous ring connected to the rim, the ring being selected from the group consisting of a gas-containing flexible tube, a liquid-containing flexible tube, a flexible foam ring, a semi-flexible foam ring and an integral skin foam ring; and, (c.) a plastic handle connected to the base.

In some preferred embodiments, the present invention is a method of treating a mammal with pulmonary secretion congestion utilizing the aforesaid pulmonary secretion mobilization device. The methodology involves repeatedly tamping the mammal on the back in a congested lung area with a pulmonary secretion mobilization device, that includes: (a.) a cup structure having a hollow inside volume and air impermeable walls and base, the cup structure having a rim in a single flat plane; (b.) an impact-receiving continuous ring connected to the rim, the ring being selected from the group consisting of a gas-containing flexible tube, a liquid-containing flexible tube, a flexible foam ring, a semi-flexible foam ring and an integral skin foam ring; and, (c.) a handle connected to the base.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 is a side, partially cut view of one preferred embodiment of a present invention pulmonary secretion mobilization device;

FIG. 2 is a bottom view of another preferred embodiment of a present invention pulmonary secretion mobilization device with the handle not shown;

FIG. 3 is a bottom view of yet another preferred embodiment of a cup structure of a present invention pulmonary secretion mobilization device;

FIG. 4 is a side view of another preferred embodiment of a present invention pulmonary secretion mobilization device;

FIG. 5 is a bottom view of another preferred embodiment of a present invention pulmonary secretion mobilization device having a polygonal periphery;

FIG. 6 is a side view of the present invention device shown in FIG. 5 above;

FIG. 7 is a bottom view of another preferred embodiment of a present invention pulmonary secretion mobilization device heart shaped, glow-in-the-dark cup structure; and,

FIG. 8 is a partially cut side view of a preferred embodiment of a present invention pulmonary secretion mobilization device that includes the cup structure shown in FIG. 7.

### DETAILED DESCRIPTION OF THE EMBODIMENTS

Referring now to the Figures, FIG. 1 illustrates one preferred embodiment present invention pulmonary secretion mobilization device 1. Device 1 has a cup structure made up of a solid dome that may be viewed as a wall 3 and a base 15. This cup structure has a hollow inside volume 9 and the wall and base are impermeable to air. The cup structure has a rim 17 that is substantially in a flat plane. It is not necessary for the rim to be perfectly flat but cannot be out of plane enough to disrupt the functionality of the impact-receiving continuous ring 5, connected thereto.

Impact-receiving continuous ring 5 is a closed hollow loop that contains a fluid 7, this fluid 7 may be any fluid that will permit movement of the ring to conform to the shape of the back of a mammal such as a person, child or infant. Thus, it may be a gas or any form of liquid, such as a pure liquid, a dispersion, a solution, an emulsion or a gel. Water is the preferred liquid and air is the preferred gas because they are substantially inert and inexpensive.

The present invention device 1 has an imaginary centerline 15 and the device in this example is axially symmetric thereto. Knob handle 13 is attached to base 15 by any known attachment mechanism, including riveting, bolting, screwing, heat welding and force fitting.

Device 1 is utilized to enhance the mobilization of undesired pulmonary secretion in the lung or lungs. Somewhat like burping a baby by tapping on the back, this present invention device is used to tamp the back at the lung areas to break up and move the undesirable pulmonary secretion, which is then more easily coughed up or otherwise expelled. The device methodology more specifically relates to rapid tapping (also called patting) on the back in the congested lung area with the cup structure facing down as shown in the Figures, so that the open area encompassed by the ring captures air that is slightly compressed by the yielding ring upon impact with the back. The ring is flexible and compressible and hence momentarily conforms to the contour of the struck area. This rapidly repeated tapping is believed to fibrillate or pulsate the lung to loosen the undesired pulmonary secretions.

FIG. 2 shows a bottom view of a similar present invention pulmonary secretion mobilization device 20. It includes the cup structure 21 and a ring 23 connected thereto (as well as a handle, not shown) for use as described above.

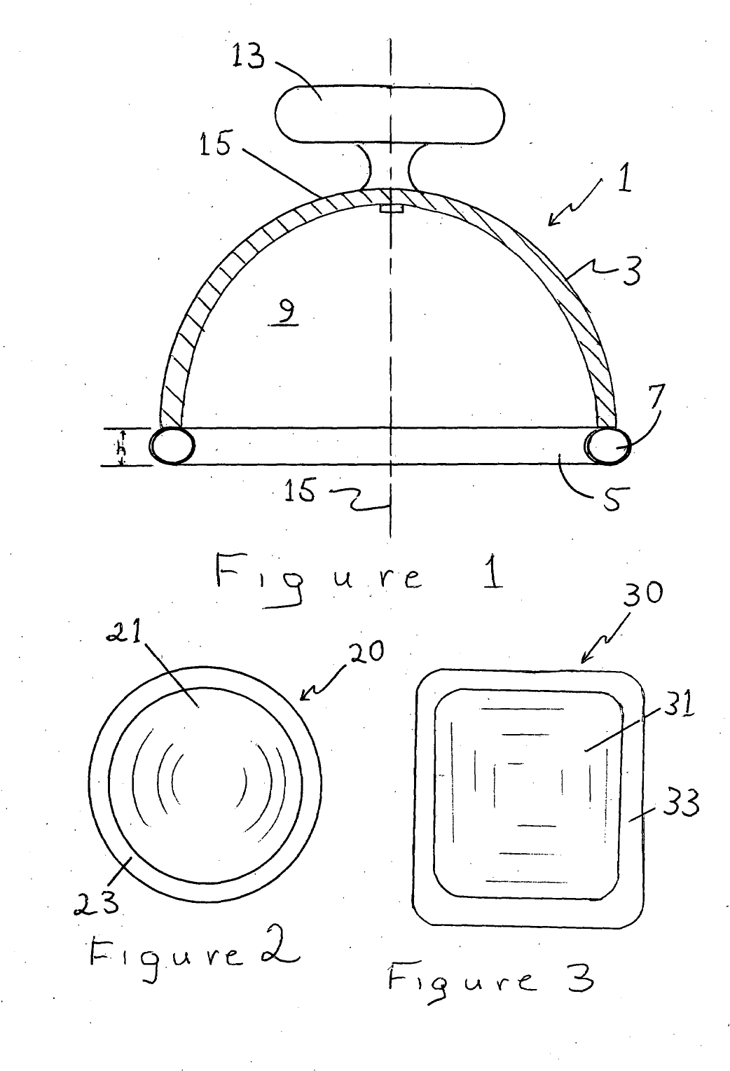

FIG. 3 is a bottom view of yet another preferred embodiment of a present invention pulmonary secretion mobilization device 30 with no handle shown. It includes a cup structure 31 having a generally rectangular shape with rounded corners. Attached to its rim is ring 33 formed of air-containing plastic continuous loop closed tube for flexibility and compressivity upon impacting the back lung area of a patient.

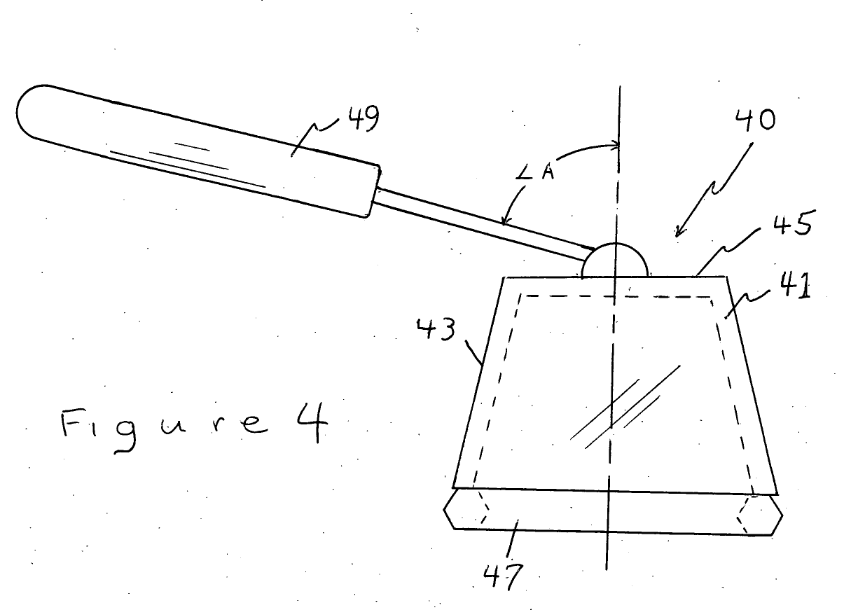

FIG. 4 is a side view of another preferred embodiment of a present invention pulmonary secretion mobilization device 40. This embodiment has four symmetric, identical sides, such as side 43, that are trapezoidal in shape. It has a base 45 and a bottom view profile that is square. There is a ring 47 containing water that is made of flexible polyethylene plastic and compresses (partially flattens) upon impact. Handle 40 is connected at an ergonomically functional angle. Thus, the cup structure has an imaginary central axis as shown and the handle 40 is an extended handle positioned at an angle of angle A, being about 60 degrees to about 90 degrees measured downwardly from the imaginary central axis, as shown.

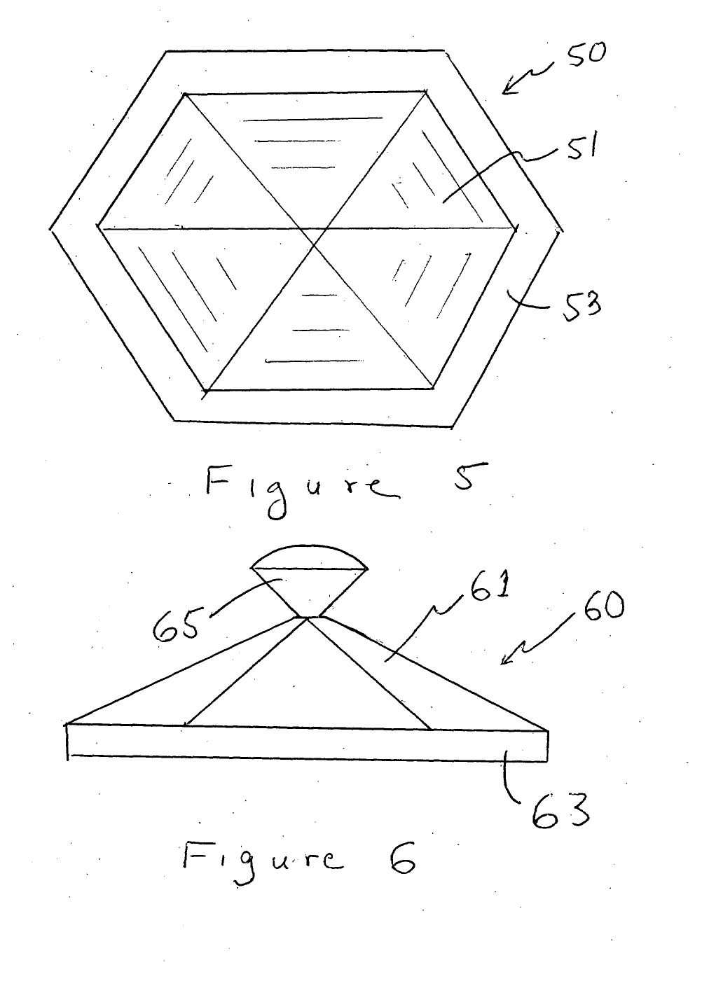

FIG. 5 is a bottom view of another preferred embodiment of a present invention pulmonary secretion mobilization device 50 having an asymmetrical polygonal periphery, a cup structure 51 and a ring 53 having the ring characteristics described in the Summary above. FIG. 6 is a side view of the present invention device 50 shown in FIG. 5 above, with identical components identically numbered. Also, knob handle 65 is shown in this Figure. This device 60 functions in the same manner as those previously described above.

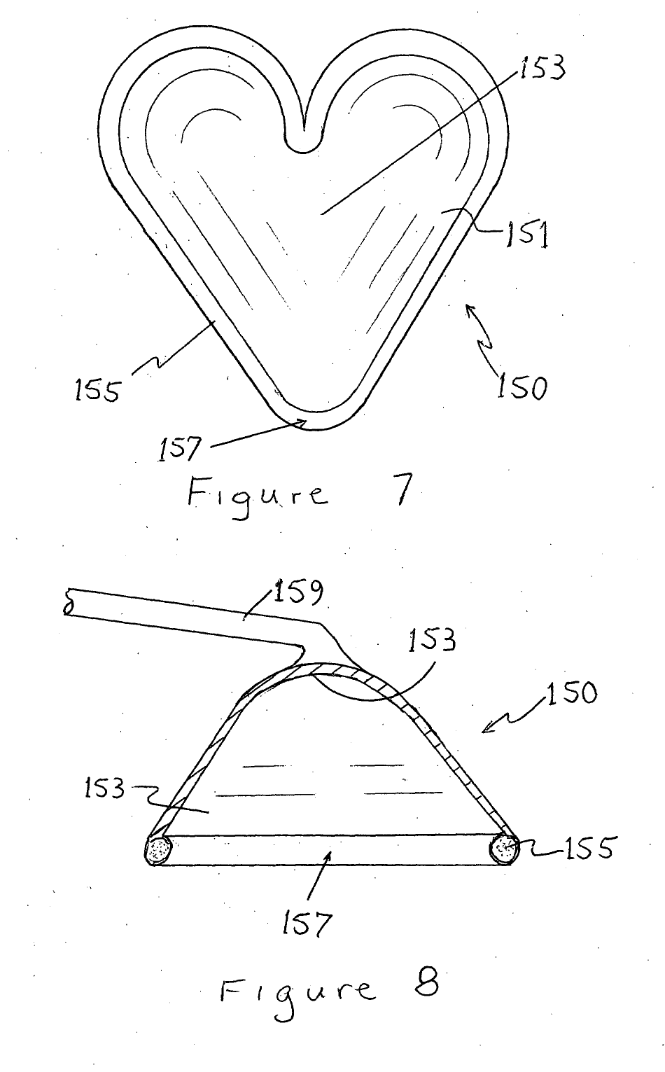

FIG. 7 is a bottom view of another preferred embodiment of a present invention pulmonary secretion mobilization device 150 and FIG. 8 is a partially cut side view of the preferred embodiment present invention pulmonary secretion mobilization device 150 that includes the cup structure shown in FIG. 7. Discussing FIGS. 7 and 8 collectively, there is shown a heart-like domed cup structure that has a base 153 and wall 151. There is a foam-containing plastic ring 157, with flexible or semi-flexible foam 155, and extended handle 159, as shown. In some preferred embodiments, this device 150 may be unistructurally formed, that is, may entirely be molded in a single step. For example, this device 150 could be formed of integral skin foam wherein the mold at the ring area is heated or cooled differently to effect the formation of the foam in the ring area only, with the cup structure and the handle having no or only micro-porous foam. The device 150 is utilized in a fashion similar to the above-described devices to rapidly pat the back lung area of a patient to mobilize pulmonary secretion.

To summarize, the present invention thus provides an easy and simple device and technique that may save lives and will certainly relieve discomfort for patients having pulmonary secretion congestion. Respiratory compromised patients that may benefit include patients with tuberculosis, pneumonia, bronchitis, paralysis, elderly, intensive care patients, including newborns.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

What is claimed is:

1. A pulmonary secretion mobilization device, which comprises:
(a.) a cup structure having a hollow inside volume and air impermeable walls and base, said cup structure having a rim in a single flat plane;
(b.) an impact-receiving continuous ring connected to said rim, said ring being selected from the group consisting of a gas-containing flexible tube, a liquid-containing flexible tube, a flexible foam ring, a semi-flexible foam ring and an integral skin foam ring; and,
(c.) a handle connected to said base.

2. The pulmonary secretion mobilization device of claim 1 wherein said handle is a knob handle.

3. The pulmonary secretion mobilization device of claim 1 wherein said handle is an extended handle protruding outwardly from said base.

4. The pulmonary secretion mobilization device of claim 1 wherein said cup structure has an imaginary central axis and said handle is an extended handle positioned at an angle of about 60 degrees to about 90 degrees measured downwardly from said imaginary central axis.

5. The pulmonary secretion mobilization device of claim 1 wherein said cup structure is symmetrical relative to an imaginary central axis.

6. The pulmonary secretion mobilization device of claim 1 wherein said cup structure rim has a footprint selected from the group consisting of a circle, an oval, a square, a rectangle and polygon having at least 5 sides.

7. The pulmonary secretion mobilization device of claim 1 wherein said ring is a gas-containing flexible tube made of plastic and containing air.

8. A pulmonary secretion mobilization device which comprises:
(a.) a plastic cup structure having a hollow inside volume and air impermeable walls and base, said cup structure having a rim in a single flat plane;
(b.) an impact-receiving continuous ring connected to said rim, said ring being selected from the group consisting of a gas-containing flexible tube, a liquid-containing flexible tube, a flexible foam ring, a semi-flexible foam ring and an integral skin foam ring; and,
(c.) a plastic handle connected to said base.

9. The pulmonary secretion mobilization device of claim 8 wherein said handle is a knob handle.

10. The pulmonary secretion mobilization device of claim 8 wherein said handle is an extended handle protruding outwardly from said base.

11. The pulmonary secretion mobilization device of claim 8 wherein said cup structure has an imaginary central axis and said handle is an extended handle positioned at an angle of about 60 degrees to about 90 degrees measured downwardly from said imaginary central axis.

12. The pulmonary secretion mobilization device of claim 8 wherein said cup structure is symmetrical relative to an imaginary central axis.

13. The pulmonary secretion mobilization device of claim 8 wherein said cup structure rim has a footprint selected from the group consisting of a circle, an oval, a square, a rectangle and polygon having at least 5 sides.

14. The pulmonary secretion mobilization device of claim 8 wherein said ring is a gas-containing flexible tube made of plastic and containing air.

15. A method of treating a mammal with pulmonary secretion congestion, which comprises:
repeatedly tamping said mammal on the back in a congested lung area with a pulmonary secretion mobilization device that includes:
(a.) a cup structure having a hollow inside volume and air impermeable walls and base, said cup structure having a rim in a single flat plane;
(b.) an impact-receiving continuous ring connected to said rim, said ring being selected from the group consisting of a gas-containing flexible tube, a liquid-containing flexible tube, a flexible foam ring, a semi-flexible foam ring and an integral skin foam ring; and,
(c.) a handle connected to said base.

16. The method of treating a mammal with pulmonary secretion congestion of claim 15 wherein said handle is selected from the group consisting of a knob handle and an extended handle protruding outwardly from said base.

17. The method of treating a mammal with pulmonary secretion congestion of claim 15 wherein said cup structure is symmetrical relative to an imaginary central axis.

18. The method of treating a mammal with pulmonary secretion congestion of claim 15 wherein said cup structure rim has a footprint selected from the group consisting of a circle, an oval, a square, a rectangle and polygon having at least 5 sides.

19. The method of treating a mammal with pulmonary secretion congestion of claim 15 wherein said ring is a gas-containing flexible tube containing air.

20. The method of treating a mammal with pulmonary secretion congestion of claim 15 wherein said ring is a urethane foam ring.
